(19) United States
(12) Patent Application Publication
(10) Pub. No.: US 2026/0216369 A1
(43) Pub. Date: Jul. 30, 2026

(54) RETINA-TARGETING ADENO-ASSOCIATED VIRUS VARIANT KAP AND USE THEREOF

(71) Applicant: UIF (University Industry Foundation), Yonsei University, Seoul (KR)

(72) Inventors: Jae-Hyung JANG, Seoul (KR); Junwon LEE, Seoul (KR); Jinyoung KIM, Seoul (KR); Subin CHO, Seoul (KR); Han JEONG, Seoul (KR)

(73) Assignee: UIF (University Industry Foundation), Yonsei University, Seoul (KR)

(21) Appl. No.: 19/457,285

(22) Filed: Jan. 23, 2026

Related U.S. Application Data
(60) Provisional application No. 63/749,138, filed on Jan. 24, 2025.

(30) Foreign Application Priority Data
Aug. 13, 2025 (KR) 10-2025-0112553

(51) Int. Cl. A61K 48/00; A61K 9/00; C07K 14/005; C12N 15/86

(52) U.S. Cl. CPC A61K 48/0041; A61K 9/0048; A61K 48/0075; C07K 14/005; C12N 15/86; C12N 2750/14122; C12N 2750/14143; C12N 2750/14145

(57) ABSTRACT

Disclosed is an adeno-associated virus (AAV) capsid protein variant having improved infectivity and transduction efficiency into target cells, and uses thereof. Specifically, disclosed is an AAV2 variant capsid protein selected by directed evolution for gene delivery into retinal cells, a recombinant AAV vector including the same, and uses thereof as a gene delivery vehicle for retinal cells.

### CROSS-REFERENCE TO RELATED APPLICATION

This application is based on and claims priority under 35 U.S.C. § 119 to U.S. Provisional Application No. 63/749,138, filed on Jan. 24, 2025, and to Korean Patent Application No. 10-2025-0112553, filed on Aug. 13, 2025, the disclosures of which are incorporated by reference herein in their entirety.

### INCORPORATION BY REFERENCE OF SEQUENCE LISTING

The instant application contains a Sequence Listing which has been filed electronically in xml format and is hereby incorporated by reference in its entirety. Said xml copy, created on Jan. 22, 2026, is named Q317626_SEQLIST-ST26.xml and is 7,498 bytes in size.

### BACKGROUND

### 1. Field

Disclosed is an adeno-associated virus (AAV) capsid protein variant having improved infectivity and transduction efficiency toward target cells, and uses thereof. Specifically, disclosed is an AAV2 variant capsid protein selected by directed evolution for gene delivery to retinal photoreceptor cells via intravitreal injection, a recombinant AAV vector including the same, and uses thereof as a gene delivery vehicle for retinal cells.

This research was conducted with the support of the Samsung Science & Technology Foundation (Project No. SRFC-MA2022-08).

### 2. Description of the Related Art

Gene therapy is a method of treating hereditary diseases by introducing a normal gene into human cells to correct or compensate for diseases caused by genetic defects and abnormalities, and has become a key means for treating various hereditary diseases. Adeno-associated viruses (AAVs) are gene delivery vectors widely used in the field of gene therapy because they may infect a wide range of tissues, are non-pathogenic, and have low immunogenicity.

Luxturna is an AAV2-based gene therapy product first approved by the U.S. FDA, and targets the retinal pigment epithelial (RPE) through subretinal injection. Since the subretinal space is located between the neural retina and the RPE, subretinal injection is known as a method capable of effectively targeting photoreceptor cells and the RPE. However, subretinal injection requires a complex surgical approach under general anesthesia or local anesthesia, and thus carries a risk of serious side effects such as retinal detachment, intraocular hemorrhage, and macular hole formation. In addition, because gene delivery is limited around the injection site, there is a limitation in achieving gene delivery over a wide retinal area.

Accordingly, there is a growing need for intravitreal injection, which enables less invasive and more widespread gene delivery. For gene therapy via intravitreal injection, vectors having higher tissue specificity and higher gene delivery and expression efficiency than prior AAV vectors are required. To achieve this, AAV capsid engineering has been actively studied.

Directed evolution is a widely used high-throughput screening method for engineering improved biomolecules, and is a technique that mimics natural selection by repeating processes of inducing/introducing genetic variations and selection. AAV capsid directed evolution is conducted by introducing mutations into a wild-type AAV capsid gene to construct an AAV capsid library of diverse sequences, and screening this library for capsid variants having desired properties, to thereby obtain new capsid variants.

Through such AAV capsid engineering, recombinant AAV vectors having high tissue specificity and gene transduction efficiency have been developed and utilized (Korean Patent Registration No. 2234930), in particular, hereditary retinal diseases are often single-gene diseases, and are thus considered promising indications for gene therapy. Accordingly, AAV variants such as AAV2-7m8, AAV2.GL, and AAV2.NN have been developed (Science Translational Medicine, 5 (189), 189ra76 and EMBO Molecular Medicine, 13 (4), e13392). However, there remains a need for AAV variants having superior transduction capability that may be delivered via intravitreal injection to induce gene expression in desired retinal cells with high specificity.

Accordingly, the inventors of the present disclosure completed the disclosure by selecting, from an AAV library constructed by directed evolution, an AAV2 variant exhibiting excellent gene delivery into photoreceptor cells via intravitreal injection.

### SUMMARY

Provided is an AAV2 variant capsid protein having gene delivery capability to photoreceptor cells through intravitreal injection.

In addition, provided is a recombinant AAV2 vector including an AAV2 variant capsid protein having gene delivery capability to photoreceptor cells through intravitreal injection.

Furthermore, provided is a use of a recombinant AAV2 vector including an AAV2 variant capsid protein having gene delivery capability to photoreceptor cells through intravitreal injection for treatment of ocular diseases.

Additional aspects will be set forth in part in the description, which follows and, in part, will be apparent from the description, or may be learned by practice of the presented embodiments of the disclosure.

According to an aspect of the disclosure, provided is an AAV2 variant capsid protein including a peptide KAPPNNP inserted into a GH-loop of a wild-type AAV2 capsid protein.

The AAV2 variant capsid protein of the disclosure was obtained by selecting an AAV2 having tropism for retinal cells, specifically photoreceptor cells, through intravitreal injection, by engineering via directed evolution of a wild-type AAV2 capsid protein, and by clarifying its structure and gene delivery activity. The AAV variant capsid protein of the disclosure may be used to deliver a target gene to photoreceptor cells through intravitreal injection. For an AAV2 variant to target photoreceptor cells through intravitreal injection, it must penetrate the inner limiting membrane (ILM) and the inner retina, and since the target photoreceptor cells are widely distributed throughout the outer retina of the entire retina, gene delivery across the entire retina must be possible to achieve a sufficient therapeutic effect.

According to an aspect of the disclosure, the peptide KAPPNNP may be inserted at amino acid 587 of the wild-type AAV2 capsid protein of SEQ ID NO: 1 or at a position corresponding thereto.

The AAV2 variant capsid protein of the disclosure includes an insertion of the peptide KAPPNNP between amino acid N587 and amino acid R588 of the wild-type AAV2 capsid protein of SEQ ID NO: 1, and was confirmed to deliver a target gene to retinal cells including photoreceptor cells, ganglion cells, Müller glial cells, and bipolar cells, with high efficiency via intravitreal injection, as compared with wild-type AAV2 and AAV2.7m8 (having insertion of the peptide “LGETTRP” between N587 and R588 of wild-type AAV2: SEQ ID NO: 3) and AAV2.NN (having insertion of the peptide “NNPTPSR” between N587 and R588 of wild-type AAV2: SEQ ID NO: 4), which are known to have gene delivery capability to retinal photoreceptor cells, and was named “AAV2.KAP” or an “KAP variant.”

The wild-type AAV2 capsid protein includes the amino acid sequence of SEQ ID NO: 1. The KAP variant according to the disclosure includes, relative to the wild-type AAV2 capsid protein, KAPPNNP inserted into the GH loop, between N587 and R588 in the amino acid sequence of SEQ ID NO: 1.

According to an aspect of the disclosure, the AAV2 variant capsid protein may be composed of the amino acid sequence of SEQ ID NO: 2 or an amino acid sequence having 85% or more, 90% or more, or 95% or more sequence identity thereto.

As used herein, the term “AAV” is an abbreviation of adeno-associated virus, and refers to the virus itself or a derivative thereof. Unless otherwise specified, AAV includes all subtypes thereof, naturally occurring forms, and recombinant forms thereof. AAV is a non-pathogenic parvovirus consisting of a single-stranded DNA genome of 4.7 kb in length within a non-enveloped icosahedral capsid. The genome includes three open reading frames (ORFs) flanked by inverted terminal repeats (ITRs) that function as viral origins of replication and packaging signals the rep ORF encodes four non-structural proteins that play roles in viral replication, transcriptional regulation, site-specific integration, and virion assembly, and the cap ORF encodes three structural proteins (VP1-3) that assemble to form a 60-mer viral capsid, and an ORF present as an alternative reading frame within the cap gene encodes an assembly-activating protein (AAP), which is a viral protein that localizes AAV capsid proteins to the nucleolus and functions in the capsid assembly process.

The genome sequences and native terminal repeat (terminal repeat: TR) sequences of various serotypes of AAV, and Rep proteins and capsid subunits thereof, are known in the art. These sequences may be found in the literature or in public databases such as GenBank. For example, the genome sequence of AAV2 is registered under GenBank Accession NC_001401.

As used herein, the term “tropism” refers to preferential targeting of cells of a particular host species or a particular cell type within a host species by a virus (for example, AAV), and is used interchangeably with “taxis.” For example, a virus that may infect retinal cells with high specificity has a higher tropism for retinal cells than for non-retinal cells. Since the capsid protein of an AAV virus determines infectivity toward target cells or tissues, tropism or taxis are used as a selection pressure to develop AAV variants that may deliver genes to target cells or tissues with high specificity and efficiency through capsid engineering via directed evolution.

As used herein, the term “retinal cells” refers to all types of cells present in the retina, and include, for example, retinal ganglion cells, bipolar cells, photoreceptor cells, Müller glial cells, horizontal cells, amacrine cells, astrocytes, microglia, retinal vascular cells, and retinal pigment epithelial cells (retinal pigment epithelium: RPE), but are not limited thereto.

As used herein, the term “gene delivery capability” refers to the ability or activity of an AAV vector to specifically deliver and express a gene in target cells or tissues, and is used interchangeably herein with “gene transduction efficacy,” “infectivity” or “transduction efficacy.”

As used herein, the term “corresponding position” refers to a position corresponding through sequence alignment with respect to a specific position, or a position determined to perform the same or substantially the same function.

As used herein, the term “sequence identity” refers to a ratio at which the type of amino acid or base at each specific position matches between polypeptides or polynucleotides being compared, and may be used interchangeably with “homology” with respect to sequences. For example, calculation of homology (%) between two polypeptide sequences is performed by optimally aligning the two sequences for comparison purposes and comparing amino acids at corresponding amino acid positions, and if the same amino acid is present at a position of the first sequence and the corresponding position of the second sequence, the two sequences are determined to be identical at that position. Sequence homology may be determined using software such as BLASTP, BLASTN, and FASTA.

According to an aspect of the disclosure, the AAV2 variant capsid protein may confer increased infectivity to retinal cells as compared with a wild-type AAV2 capsid protein.

According to an aspect of the disclosure, the retinal cells may be selected from the group consisting of retinal ganglion cells, bipolar cells, photoreceptor cells, Müller glial cells, horizontal cells, amacrine cells, astrocytes, microglia, retinal vascular cells, and retinal pigment epithelial cells.

According to an aspect of the disclosure, the AAV2 variant capsid protein may confer increased infectivity to photoreceptor cells as compared with a wild-type AAV2 capsid protein.

According to an aspect of the disclosure, the AAV2 variant capsid protein may confer increased infectivity to photoreceptor cells by intravitreal injection as compared with a wild-type AAV2 capsid protein.

According to another aspect of the disclosure, provided is an isolated nucleic acid encoding an AAV variant capsid protein.

According to an aspect of the disclosure, the isolated nucleic acid may include a nucleotide sequence encoding the amino acid sequence of SEQ ID NO: 2 or a nucleotide sequence having 95% or more sequence homology thereto.

According to another aspect of the disclosure, provided is a host cell including an isolated nucleic acid encoding an AAV2 variant capsid protein including a peptide KAPPNNP inserted at amino acid 587 of a wild-type AAV2 capsid protein of SEQ ID NO: 1 or at a position corresponding thereto.

The host cell may be used to produce a recombinant AAV virion including an AAV2 variant capsid protein. In this case, the host cell is referred to as a “packaging cell.” The host cell may be selected from various cells including, but not limited to, HEK293 cells, HEK293T cells, Hela cells, Sf9 cells, 293A cells, 293H cells, etc.

According to an aspect of the disclosure, the isolated nucleic acid is stably or transiently introduced into a host cell using methods including, but not limited to, electroporation, calcium phosphate precipitation, liposome-mediated transfection, and the like.

According to another aspect of the disclosure, provided is an AAV2 variant capsid protein including a peptide KAPPNNP inserted at amino acid 587 of a wild-type AAV2 capsid protein of SEQ ID NO: 1 or at a position corresponding thereto, and

a recombinant AAV vector including a heterologous nucleic acid including a sequence encoding a target product.

As used herein, the term “recombinant AAV vector” refers to an AAV vector including a polynucleotide sequence that is not derived from AAV, that is, a polynucleotide sequence that is heterologous with respect to AAV, and is used interchangeably with “rAAV.” Typically, in a recombinant AAV vector, the heterologous polynucleotide sequence is a nucleic acid sequence encoding a target product to be delivered to a target cell.

As used herein, the term “heterologous” refers to a source that is genetically distinct from an AAV2 vector, for example, a nucleic acid originating from a different species.

According to an aspect of the disclosure, the recombinant AAV vector may exhibit increased transduction into retinal tissues/cells as compared with an AAV vector (AAV2 vector) including a wild-type AAV2 capsid protein.

According to an aspect of the disclosure, the recombinant AAV vector may exhibit increased transduction into retinal tissues/cells through intravitreal injection as compared with an AAV vector (AAV2 vector) including a wild-type AAV2 capsid protein.

The recombinant AAV vector according to an embodiment may be used to prepare a recombinant AAV virion. When introduced into a suitable host cell, the recombinant AAV vector may provide production of a corresponding recombinant AAV virion.

According to an aspect of the disclosure, the target product may be a polypeptide or a nucleic acid. The target product refers to a product that may supplement or enhancing a function that is deficient or insufficient in a subject, or reducing or eliminating a defective function, thereby may bring about a therapeutic effect for a target disease. For example, the target product may be a gene product or its activity that is directly or indirectly related to an ocular disease such as a retinal disease, or may be one that enhances the function of retinal cells, and may be, for example, pigment epithelium-derived factor (PEDF), or rhodopsin.

According to an aspect of the disclosure, the target product may be a therapeutic nucleic acid and may target any gene associated with an ocular disease, such as achromatopsia, macular degeneration such as wet age-related macular degeneration (AMD) or dry AMD, cataracts, pan choroidal atrophy, glaucoma, optic neuropathy, Marfan syndrome, myopia, nodular choroidal angiopathy, retinitis pigmentosa, Stargardt disease, Usher syndrome, Leber congenital amaurosis, Leber hereditary optic neuropathy, uveal melanoma, or X-linked retinoschisis or any hereditary disease occurring in ocular tissue. The target gene may be, a gene encoding a protein selected from the group consisting of, for example, VEGFA, VEGFB, ANGPT2, FLT1, KDR, PDGFB, CFH, ARMS2, HTRA1, TIMP3, C3, C2, CFB, SERPINF1, ANGPT1, CFI, CD59, ABCA1, ABCA4, RHO, RPGR, PRPF3, PRPF31, PRPF8, PDE6A, PDE6B, PDE6G, CNGB1, CNGA1, AIPL1, CRX, CRB1, CEP290, RS1, USH2A, MYO7A, USH1C, CLRN1, BBS1, BBS10, BEST1, PROM1, ELOVL4, PRPH2, RDH12, RPE65, CDH3, PCDH15, GUCY2D, GUCA1A, TULP1, LRAT, GNAT2, CNGB3, CNGA3, PDE6C, PDE6H, ATF6, GRM6, GPR179, TRPM1, NYX, CABP4, NR2E3, NRL, IMPDH1, IMPDH2, SNRNP200, HK1, GNAT1, KCNV2, RAX2, CHM, MERTK, EYS, NMNAT1, RP1, RP2, LCA5, RD3, RLBP1, MT-ND4, MT-ND1, MT-ND6, NDP, LRP5, TSPAN12, FZD4, CAPN5, TEK, CYP1B1, CYP4V2, MYOC, OPTN, OPA1, IL10, IL1RN, TNFRSF1A, HGF, PEDF, sFLT1, STNFR, GNAQ, GNA11, BAP1, SF3B1, EIF1AX, TP53, TNFSF10, DDEF1, CDKN2A, p14ARF, IFNB1, HSV-TK, BDNF, GDNF, CNTF, NTF3, NGF, XIAP, SOD2, TXN, NAMPT, SLC16A8, GEMIN4, CYP51A1, RIC1, TAPT1, TAFIA, WDR87, APE1, MIP, GJA3, GJA8, CRYAA, CRYBB2, PRX, POLR3B, XRCC1, ZNF350, EPHA2, REP1, CALM2, MPP7, LOX1, CAV1, CAV2, PITX2, FOXC1, PAX6, LTBP2, FBN1, TGFBR2, MTHFR, MTR, MTRR, MET, UMODL1, MMP1, MMP2, CBS, IGF1, UHRF1BP1L, PTPRR, PPFIA2, P4HA2, SERPING1, FGD6, ABCG1, LOC387715, CETP, PTEN, and HERC2/OCA2, but is not limited thereto.

According to an aspect of the disclosure, the target product may be a therapeutic protein or polypeptide, and may be selected from the group consisting of, for example, VEGFA, VEGFB, ANGPT2, FLT1, KDR, PDGFB, CFH, ARMS2, HTRA1, TIMP3, C3, C2, CFB, SERPINF1, ANGPT1, CFI, CD59, ABCA1, ABCA4, RHO, RPGR, PRPF3, PRPF31, PRPF8, PDE6A, PDE6B, PDE6G, CNGB1, CNGA1, AIPL1, CRX, CRB1, CEP290, RS1, USH2A, MYO7A, USH1C, CLRN1, BBS1, BBS10, BEST1, PROM1, ELOVL4, PRPH2, RDH12, RPE65, CDH3, PCDH15, GUCY2D, GUCA1A, TULP1, LRAT, GNAT2, CNGB3, CNGA3, PDE6C, PDE6H, ATF6, GRM6, GPR179, TRPM1, NYX, CABP4, NR2E3, NRL, IMPDH1, IMPDH2, SNRNP200, HK1, GNAT1, KCNV2, RAX2, CHM, MERTK, EYS, NMNAT1, RP1, RP2, LCA5, RD3, RLBP1, MT-ND4, MT-ND1, MT-ND6, NDP, LRP5, TSPAN12, FZD4, CAPN5, TEK, CYP1B1, CYP4V2, MYOC, OPTN, OPA1, IL10, IL1RN, TNFRSF1A, HGF, PEDF, sFLT1, STNFR, GNAQ, GNA11, BAP1, SF3B1, EIF1AX, TP53, TNFSF10, DDEF1, CDKN2A, p14ARF, IFNB1, HSV-TK, BDNF, GDNF, CNTF, NTF3, NGF, XIAP, SOD2, TXN, NAMPT, SLC16A8, GEMIN4, CYP51A1, RIC1, TAPT1, TAFIA, WDR87, APE1, MIP, GJA3, GJA8, CRYAA, CRYBB2, PRX, POLR3B, XRCC1, ZNF350, EPHA2, REP1, CALM2, MPP7, LOX1, CAV1, CAV2, PITX2, FOXC1, PAX6, LTBP2, FBN1, TGFBR2, MTHFR, MTR, MTRR, MET, UMODL1, MMP1, MMP2, CBS, IGF1, UHRF1BP1L, PTPRR, PPFIA2, P4HA2, SERPING1, FGD6, ABCG1, LOC387715, CETP, PTEN, and HERC2/OCA2, but is not limited thereto.

According to another aspect of the disclosure, provided is a use of the above-described recombinant AAV vector in the treatment of an ocular disease.

According to an aspect of the disclosure, provided is a pharmaceutical composition for treatment of an ocular disease, including the above-described recombinant AAV vector.

According to an aspect of the disclosure, provided is a method of treating an ocular disease, including administering a therapeutically effective amount of the above-described recombinant AAV vector to a subject in need of treatment of an ocular disease.

As used herein, the term “ocular disease” refers to a disease related to the eye or a part or specific region of the eye, for example, the retina or choroid.

According to an aspect of the disclosure, the ocular disease may be a retinal disease, a choroidal disease, or a retinal/choroidal disease.

According to an aspect of the disclosure, the ocular disease may be selected from retinal vascular disease, optic neuropathy, hereditary retinal disease, hereditary choroidal disease, and intraocular tumor.

According to an aspect of the disclosure, the ocular disease may be selected from retina-vitreous-choroid related diseases, retinal vascular diseases, macular diseases, hereditary retinal diseases, hereditary vitreous diseases, hereditary choroidal diseases, intraocular inflammatory diseases, intraocular tumors, glaucoma, and optic neuropathies.

According to an aspect of the disclosure, the ocular disease may be, occlusion of retinal vessels including age-related macular degeneration, diabetic retinopathy, retinal artery/vein occlusion, and macular telangiectasia, optic neuropathy including glaucoma, hereditary retinal/choroidal diseases including uveitis, Leber congenital amaurosis, retinitis pigmentosa, Usher syndrome, Stargardt's Disease, Best's Disease, X-linked retinoschisis, congenital stationary night blindness, choroideremia, achromatopsia, cone dystrophy, gyrate atrophy, and Bardet-Biedl syndrome, and intraocular tumors such as uveal melanoma, intraocular lymphoma, and retinoblastoma, but are not limited thereto.

According to an aspect of the disclosure, the ocular disease may be selected from macular degeneration, diabetic retinopathy, occlusion of retinal vessels, macular telangiectasia, retinopathy of prematurity, myopic degeneration, retinitis pigmentosa, Leber congenital amaurosis, Best's Disease, Stargardt's Disease, congenital stationary night blindness, X-linked retinoschisis, Bietti crystalline dystrophy, achromatopsia, cone dystrophy, cone-rod dystrophy, maculopathy, Usher syndrome, Bardet-Biedl syndrome and other syndromic retinitis pigmentosa, pan choroidal atrophy, central areolar choroidal dystrophy, gyrate atrophy of choroid and retina, glaucoma, other optic neuropathies, uveitis, uveal melanoma, intraocular lymphoma, retinoblastoma, retinoschisis, and other retinal injury.

According to an aspect of the disclosure, the recombinant AAV vector may be administered by intravitreal injection, subretinal injection, intraocular injection through suprachoroidal injection, or any suitable route of administration for delivery to the retina, including, but not limited to, periocular, intravenous, intraarterial, intranasal administration, etc.

According to an aspect of the disclosure, the recombinant AAV vector may be administered by intravitreal injection. The recombinant AAV vector of the disclosure, when administered via intravitreal injection, may migrate through the vitreous humor, pass through the ILM, and migrate through the retinal layers more efficiently than an AAV vector including a wild-type AAV2 capsid protein.

According to an aspect of the disclosure, the recombinant AAV vector may be administered by intraocular injection.

The recombinant AAV vector according to the disclosure has an increased ability, when administered by intravitreal injection, to deliver and express a heterologous nucleic acid to retinal cells across the ILM as compared with an AAV2 vector including a wild-type capsid protein.

The recombinant AAV vector according to the disclosure may cross the ILM and may traverse cell layers including Müller glial cells, bipolar cells, etc. to reach photoreceptor cells. For example, when administered via intravitreal injection, the recombinant AAV vector according to the disclosure may cross the ILM and may traverse cell layers including Müller glial cells, bipolar cells, etc. to reach photoreceptor cells.

In some embodiments, the recombinant AAV vector may specifically infect retinal cells, and in particular, may specifically infect photoreceptor cells.

As used herein, the term “therapeutically effective amount” refers to an amount sufficient to alleviate (for example, ameliorate, reduce, or diminish) at least one of the symptoms associated with a disease condition. For example, a therapeutically effective amount for in vivo injection, that is, for direct injection into the retina, may range from about 106 to about 1015, for example, from about 108 to about 1012 genome copies (GC). For in vitro transduction, an effective amount of rAAV virions delivered to cells may range from about 108 to about 1013 GC. A therapeutically effective amount of the recombinant AAV vector according to the disclosure may be readily determined by a person of ordinary skill in the art in consideration of the target disease, the age, sex, and height of the subject, the severity of the disease to be treated, and the like.

According to an aspect of the disclosure, the recombinant viral vector is administered in an amount sufficient to generate infection (or transduction) and expression of the heterologous nucleic acid sequence in retinal cells of the subject.

According to an aspect of the disclosure, the retinal cells may be selected from the group consisting of photoreceptor cells, retinal ganglion cells, bipolar cells, Müller glial cells, horizontal cells, amacrine cells, astrocytes, microglial cells, retinal vascular cells, and retinal pigment epithelium (RPE) cells.

According to an aspect of the disclosure, the retinal cells are photoreceptor cells, for example, cone cells and/or rod cells.

According to an aspect of the disclosure, the retinal cells are retinal ganglion cells (RGCs).

According to an aspect of the disclosure, the retinal cells are RPE cells.

According to an aspect of the disclosure, the pharmaceutical composition may be administered one or more times over periods of various intervals, for example, daily, weekly, monthly, yearly, etc. to achieve a desired level of gene expression.

According to an aspect of the disclosure, the pharmaceutical composition may include a pharmaceutically acceptable carrier, excipient, or additive.

According to another aspect of the disclosure, provided is an in vitro method of delivering a heterologous nucleic acid encoding a target product to retinal cells, including contacting the above-described recombinant AAV vector with retinal cells.

According to the disclosure, delivery of a heterologous nucleic acid to retinal cells may be used for treatment of a retinal disease.

According to an aspect of the disclosure, the target product and retinal cells are as described above.

### BRIEF DESCRIPTION OF THE DRAWINGS

The above and other aspects, features, and advantages of certain embodiments of the disclosure will be more apparent from the following description taken in conjunction with the accompanying drawings, in which:

FIG. 1 illustrates a schematic diagram of a process for selecting an AAV2 variant capsid protein according to an embodiment by directed evolution, and the next-generation sequencing (NGS) results, wherein a heptameric peptide insertion AAV library (primary library) was constructed by inserting a random heptameric peptide sequence at position 587 of a wild-type AAV2 capsid protein (SEQ ID NO: 1), and screening was performed using human retinal organoids (CRX-tdTomato retinal organoids) and mouse (NRL-EGFP) retinas to select AAV variants having tropism for photoreceptor cells, and thereafter, cap genes were obtained from variants selected in human retinal organoids (O) and mouse photoreceptors (M), subjected to NGS analysis, and selected through primary screening, re-packaged to construct a photoreceptor-directed AAV library (secondary library), and secondary screening including transduction, NGS analysis, and selection was performed using the secondary libraries (O and M) in human retinal organoids and mouse retinas, respectively, thereby selecting AAV2 variant capsid proteins having improved transduction into photoreceptor cells as compared to AAVs having a wild-type AAV2 capsid;

FIG. 2 illustrates a vp3 structural image of AAV2.KAP including a KAP variant according to an embodiment predicted by ColabFold, in which the portion indicated by a dotted circle represents the inserted heptameric peptide;

FIG. 3A and FIG. 3B illustrate the gene delivery capability of AAV2.KAP including a KAP variant according to an embodiment, verified in human retinal organoids, in which, when recombinant AAV2.KAP (SEQ ID NO: 2), wild-type AAV2 (SEQ ID NO: 1), and improved AAV2.7m8 (SEQ ID NO: 3) and AAV2.NN (SEQ ID NO: 4), each carrying a GFP (green fluorescent protein) gene as a target gene, were transduced into human retinal organoids (CRX-tdTomato), it was confirmed that AAV2.KAP has superior gene expression efficiency throughout the retinal organoid as compared with wild-type AAV2 and the improved AAV2.7m8 and AAV2.NN; FIG. 3A is a low-magnification image, and FIG. 3B is a high-magnification image;

FIG. 4A and FIG. 4B illustrate the results of quantitative analysis of gene transduction efficiency of AAV2.KAP according to an embodiment, confirmed in human retinal organoids, in which tdTomato fluorescence labels photoreceptor cells, and GFP fluorescence labels cells to which the gene has been delivered, and in FIG. 4B, the gene transduction efficiency to all cells (Total) was analyzed as a ratio of GFP-positive cells among total cells, gene transduction efficiency to photoreceptor cells (Photoreceptor) was analyzed as a ratio of GFP-positive cells among tdTomato-positive cells, and gene transduction efficiency to non-photoreceptor cells (Non-photoreceptor) was analyzed as a ratio of GFP-positive cells among tdTomato-negative cells, and AAV2.KAP exhibited significantly higher gene transduction efficiency in total cells, photoreceptor cells, and non-photoreceptor cells of the retinal organoids than wild-type AAV2 and the improved AAV2.7m8 and AAV2.NN, and such differences were statistically significant;

FIG. 5 illustrates the results of immunofluorescence staining showing gene transduction efficiency of recombinant AAV2 vectors carrying a gene encoding GFP as a target gene in AAV2.KAP, wild-type AAV2, improved AAV2.7m8, and AAV2.NN, according to an embodiment, in which antibodies labeling photoreceptor cells and Müller glial cells (CRX and Recoverin antibodies: photoreceptor cells, CARLBP and SOX9: Müller glial cells) and an antibody against GFP fluorescence were used;

FIG. 6 illustrates the results of verifying the in vivo gene transduction efficiency of AAV2.KAP according to an embodiment, showing expression patterns of a target gene observed after intravitreal administration of AAV2.KAP carrying a tdTomato gene as a target gene, and wild-type AAV2, improved AAV2.7m8, and AAV2.NN as controls, to reporter mice (Nrl-EGFP) labeling rod cells with EGFP fluorescence, wherein in the figures, RGC refers to retinal ganglion cells, INL refers to the inner nuclear layer, ONL refers to the outer nuclear layer, and OS refers to the outer segment, and while wild-type AAV2 was confirmed to infect RGCs and some INL, AAV2.KAP was confirmed to have significantly higher expression of the delivered tdTomato gene, which was delivered not only to RGCs and INLs but also to the ONL and OS regions, which are the nuclear layers of photoreceptor cells; and

FIG. 7 illustrates the results of quantitative analysis of the in vivo gene transduction efficiency of AAV2.KAP according to an embodiment, showing expression patterns of a target gene confirmed by FACS analysis after intravitreal administration of AAV2.KAP carrying a tdTomato gene as a target gene, and wild-type AAV2, improved AAV2.7m8, and AAV2.NN as controls, to mice (Nrl-EGFP), in which EGFP fluorescence labels rod cells, and tdTomato fluorescence labels cells to which the gene has been delivered, gene transduction efficiency to the entire retinal cells (Total) was measured as a ratio of tdTomato-positive cells among total cells, gene transduction efficiency to rod cells was measured as a ratio of tdTomato-positive cells among EGFP-positive cells, and gene transduction efficiency to non-rod retinal cells was measured as a ratio of tdTomato-positive cells among EGFP-negative cells, and AAV2.KAP exhibited significantly higher gene transduction efficiency in total cells, rod photoreceptor cells, and non-rod cells than wild-type AAV2 and the improved AAV2.7m8 and AAV2.NN, and such differences were statistically significant; and

FIG. 8 illustrates the gene transduction efficiency of AAV2 variants by retinal cell type, as confirmed by immunofluorescence staining after intravitreal injection of recombinant AAV2 vectors carrying GFP as a target gene into wild-type mice (C57BL/6J), including AAV2.KAP according to an embodiment, and wild-type AAV2, improved AAV2.7m8, and AAV2.NN as control groups, in which immunofluorescence staining performed using antibodies labeling photoreceptor cells (rod and cone cells), ganglion cells, Müller glial cells, and bipolar cells, and an antibody against GFP fluorescence delivered by the recombinant AAV2 vectors, shows that AAV2.KAP exhibits higher gene transduction efficiency to photoreceptor cells, ganglion cells, Müller glial cells, and bipolar cells than wild-type AAV2, and higher gene transduction efficiency to photoreceptor cells than the improved AAV2.7m8 and AAV2.NN.

### DETAILED DESCRIPTION

Reference will now be made in detail to embodiments, examples of which are illustrated in the accompanying drawings, wherein like reference numerals refer to like elements throughout. In this regard, the present embodiments may have different forms and should not be construed as being limited to the descriptions set forth herein. Accordingly, the embodiments are merely described below, by referring to the figures, to explain aspects of the present description. As used herein, the term “and/or” includes any and all combinations of one or more of the associated listed items. Expressions such as “at least one of,” when preceding a list of elements, modify the entire list of elements and do not modify the individual elements of the list.

The disclosure will be made clear by reference to the embodiments disclosed in detail below together with the accompanying figures. However, the disclosure is not limited to the examples, but may be embodied in various forms, and the examples are provided merely to make the description of the disclosure complete and to fully convey the scope of the disclosure a person of ordinary skill in the art to which the disclosure pertains, and the disclosure is defined by the claims.

### Example 1. Selection of KAP Variant

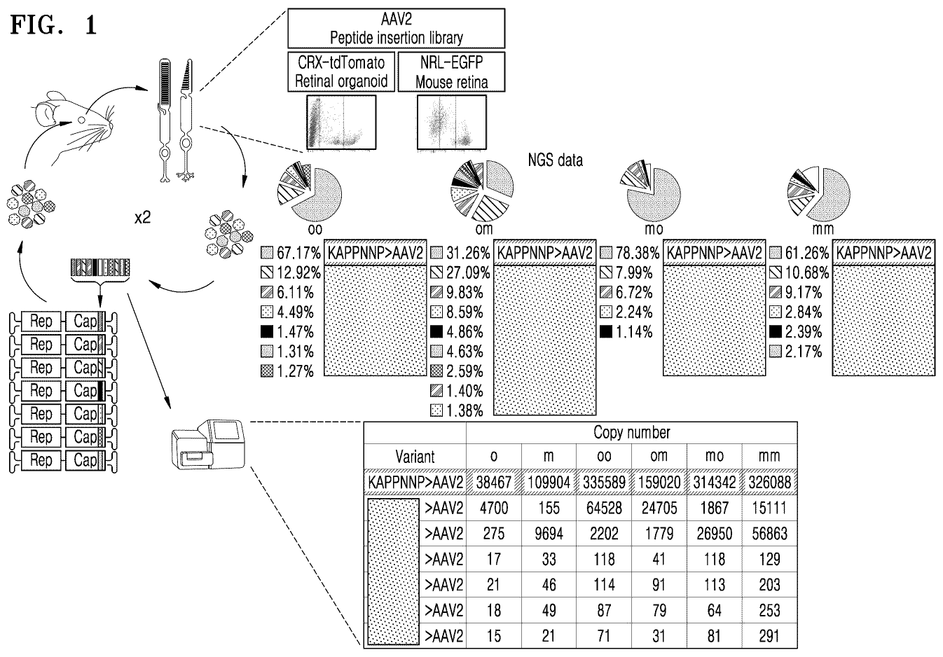

Wild-type AAV2 has difficulty delivering genes to photoreceptor cells via intravitreal injection. Accordingly, to select an AAV2 capsid variant capable of delivering and expressing a target gene in photoreceptor cells via intravitreal injection, AAV2 capsid engineering was performed by directed evolution. A schematic diagram of the directed evolution method is illustrated in FIG. 1.

In vitro and in vivo directed evolution techniques have been utilized to develop AAV variants improved over existing AAV-based gene delivery vectors. Directed evolution techniques are known in the art, and are disclosed, for example, in PCT Publication No. WO 2014/194132. Directed evolution is a capsid engineering method that mimics natural evolution through repeated processes of genetic diversification and selection, and enables accumulation of beneficial mutations that progressively improve the function of biomolecules such as AAV-based virions. In this approach, various mutations are introduced using error-prone PCR for introducing random point mutations into a wild-type AAV cap gene, DNA shuffling for generating random chimeras, random peptide insertion, transposon-mediated mutagenesis, and the like, followed by packaging of AAV particles to generate a library of AAV variants, and application of selection pressure to isolate unique variants having superior phenotypes capable of overcoming barriers to gene delivery.

In this Example, screening was performed on retinas (in vitro human-derived retinal organoids and in vivo mouse retinas) using a heptameric peptide insertion library in which random heptameric peptides were inserted at position 587 of the wild-type AAV2 capsid sequence (SEQ ID NO: 1). The position at which the heptameric peptide was inserted is known to be involved in interactions with external receptors of AAV, and thus may be involved in cell specificity and gene transduction efficiency.

### 1.1. Preparation of In Vitro Human Retinal Organoids

Human retinal organoids to be used for evaluating gene transduction efficacy of AAV2 variants to photoreceptor cells were prepared according to the method disclosed in Wahlin, K. J. et al., Photoreceptor Outer Segment-like Structures in Long-Term 3D Retinas from Human Pluripotent Stem Cells, Sci Rep 7, 766 (2017). Specifically, human retinal organoids were produced using human embryonic stem cells that express tdTomato fluorescent protein specifically in photoreceptor cells. Human embryonic stem cells expressing tdTomato fluorescent protein specifically in photoreceptor cells were cultured in mTesR1 basal medium (Stem Cell Technologies, Vancouver, Canada) on plates coated with Matrigel (Corning, NY, USA). The cells were cultured for 4-5 days prior to induction of differentiation into retinal organoids, and the cultured stem cells were dissociated using Accutase (BD Biosciences, CA, USA), followed by aggregation at a density of 3,000 cells per well using a low-cell adhesion 96-well plate (Corning) having a U-shaped bottom. Thereafter, differentiation and culture were carried out for 30 weeks or more to obtain human retinal organoids. All cultures for the preparation of human retinal organoids were maintained at 37° C. in a humidified environment with 5% carbon dioxide.

### 1.2. In Vitro and In Vivo Screening

The heptameric peptide insertion library was transduced at 5×10¹⁰ genome copies (GC) into human retinal organoids derived from reporter embryonic stem cells in which photoreceptor cells were labeled with tdTomato fluorescence, prepared in 1.1, and after 3 weeks, variants preferentially delivered to photoreceptor cells were in vitro screened by FACS sorting based on tdTomato fluorescence.

In addition, the heptameric peptide insertion library was intravitreally injected at 2×10¹⁰ GC into mice (B6.Cg-Tg (Nrl-EGFP) 1Asw/J) in which rod photoreceptor cells were labeled with EGFP fluorescence, and after 3 weeks, variants capable of infecting photoreceptor cells via intravitreal injection in the mouse retina were in vivo screened by FACS sorting based on EGFP fluorescence.

For fluorescence-activated cell sorting (FACS), human retinal organoids and mouse retinas were dissociated into single cells using a Papain Dissociation System (Worthington Biochemical Corporation, NJ, USA). FACS was performed using a BD FACSymphony S6 Cell Sorter (BD Biosciences, CA, USA). For human retinal organoids, tdTomato-positive cells/tdTomato-negative cells were separated, and for mouse retinas, EGFP-positive cells/EGFP-negative cells were separated.

The separated cells were each collected by centrifugation, and DNA was extracted using a QIAamp DNA Mini Kit (QIAGEN, 51306). PCR was performed to amplify 229 bp including the inserted heptameric peptide from the extracted DNA, and target DNA was obtained as a PCR product using a QIAquick Gel Extraction Kit (QIAGEN, 51306). According to the manufacturer's instructions, Next Generation Sequencing (NGS) analysis of the PCR product was performed using MiSeq Reagent Kits v2 (ILLUMINA, MS-102-2003).

A secondary library (organoid-secondary library (O)/mouse-secondary library (M)) was prepared from variant pools selected from photoreceptor cells of human retinal organoids/mice through primary screening, and secondary screening was performed. The organoid-secondary library and the mouse-secondary library were transduced into photoreceptor fluorescence-labeled human retinal organoids (O) and intravitreally injected into rod photoreceptor fluorescence-labeled mice (M), respectively, followed by FACS sorting based on tdTomato/EGFP and analysis by NGS. A ‘KAPPNNP’ variant (hereinafter, AAV2.KAP), which was commonly detected in a total of four samples (OO, OM, MM, and MO) and was identified as having the highest copy number within each sample, was selected. This variant was named the “KAP variant.”

The amino acid sequence (SEQ ID NO: 2) of the capsid protein of AAV2.KAP, in which the KAP variant (indicated in red) is inserted at amino acid position 587 of the AAV2 wild-type capsid protein (SEQ ID NO: 1), is as follows:

MAADGYLPDWLEDFLSEGIRQWWKLKPGPPPPKPAERHKDDSRGLVLPG
YKYLGPFNGLDKGEPVNEADAAALEHDKAYDRQLDSGDNPYLKYNHADA
EFQERLKEDTSFGGNLGRAVFQAKKRVLEPLGLVEEPVKTAPGKKRPVE
HSPVEPDSSSGTGKAGQQPARKRLNFGQTGDADSVPDPQPLGQPPAAPS
GLGTNTMATGSGAPMADNNEGADGVGNSSGNWHCDSTWMGDRVITTSTR
TWALPTYNNHLYKQISSQSGASNDNHYFGYSTPWGYFDFNRFHCHFSPR
DWQRLINNNWGFRPKRLNFKLFNIQVKEVTQNDGTTTIANNLTSTVQVF
TDSEYQLPYVLGSAHQGCLPPFPADVFMVPQYGYLTLNNGSQAVGRSSF
YCLEYFPSQMLRTGNNFTFSYTFEDVPFHSSYAHSQSLDRLMNPLIDQY
LYYLSRTNTPSGTTTQSRLQFSQAGASDIRDQSRNWLPGPCYRQQRVSK
TSADNNNSEYSWTGATKYHLNGRDSLVNPGPAMASHKDDEEKFFPQSGV
LIFGKQGSEKTNVDIEKVMITDEEEIRTTNPVATEQYGSVSTNLQRGNK
APPNNPRQAATADVNTQGVLPGMVWQDRDVYLQGPIWAKIPHTDGHFHP
SPLMGGFGLKHPPPQILIKNTPVPANPSTTFSAAKFASFITQYSTGQVS
VEIEWELQKENSKRWNPEIQYTSNYNKSVNVDFTVDTNGVYSEPRPIGT
RYLTRNL

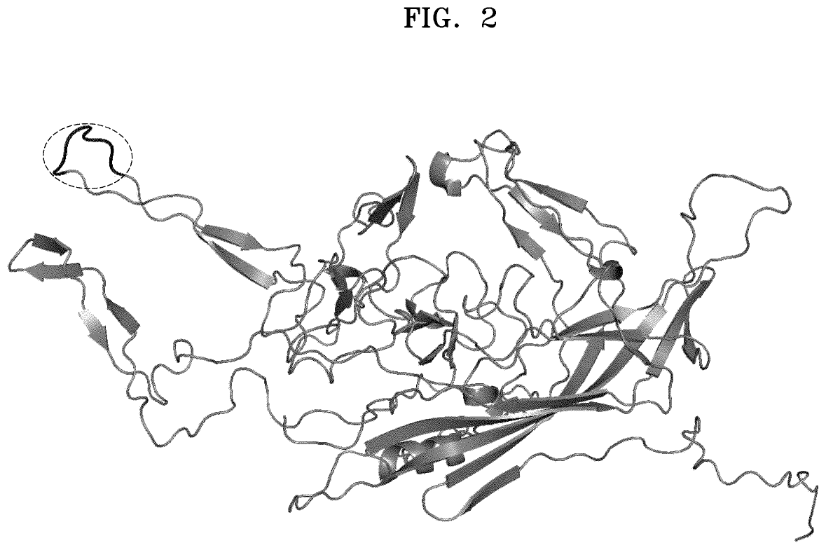

In addition, a vp3 structural image of AAV2.KAP including the KAP variant was confirmed using ColabFold (Nature Methods, 19 (6), 679-682). FIG. 2 illustrates a vp3 structural image of AAV2.KAP including a KAP variant according to an embodiment predicted by ColabFold.

### Example 2. In Vitro Validation of AAV2.KAP Variants: Human Retinal Organoids

To verify gene delivery and expression capability of the AAV2.KAP variant to photoreceptor cells selected in Example 1, expression and delivery of a target gene were confirmed in human retinal organoids.

### 2.1. Confirmation of Gene Delivery and Expression by Fluorescence Microscopy

As disclosed in Example 1, retinal organoids including photoreceptor reporters (CRX-tdTomato) that express tdTomato fluorescence specifically in photoreceptor cell were transduced with recombinant vectors AAV2.KAP-EGFP, wild-type AAV2-EGFP, and positive controls AAV2.NN-EGFP and AAV2.7m8-EGFP (see Science Translational Medicine, 5 (189), 189ra76 and EMBO Molecular Medicine, 13 (4), e13392), each carrying a gene encoding EGFP, at 5×10¹⁰ GC, and gene delivery was confirmed.

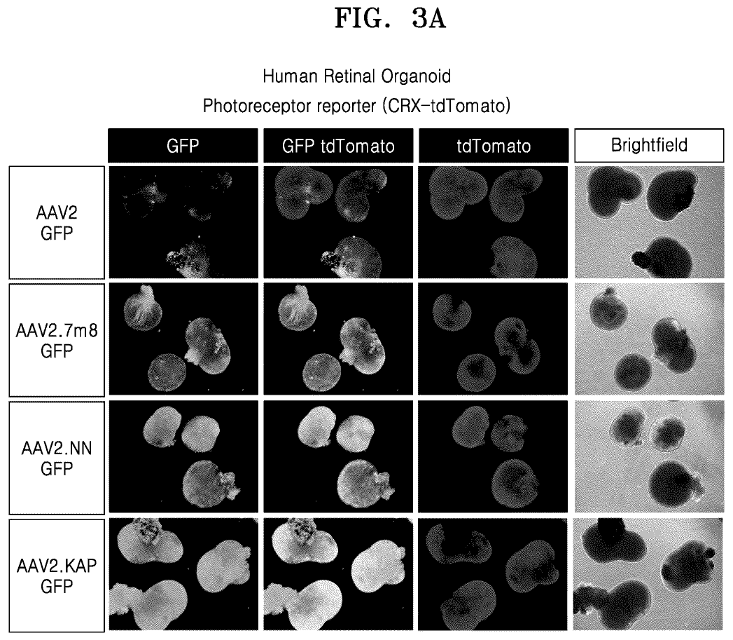

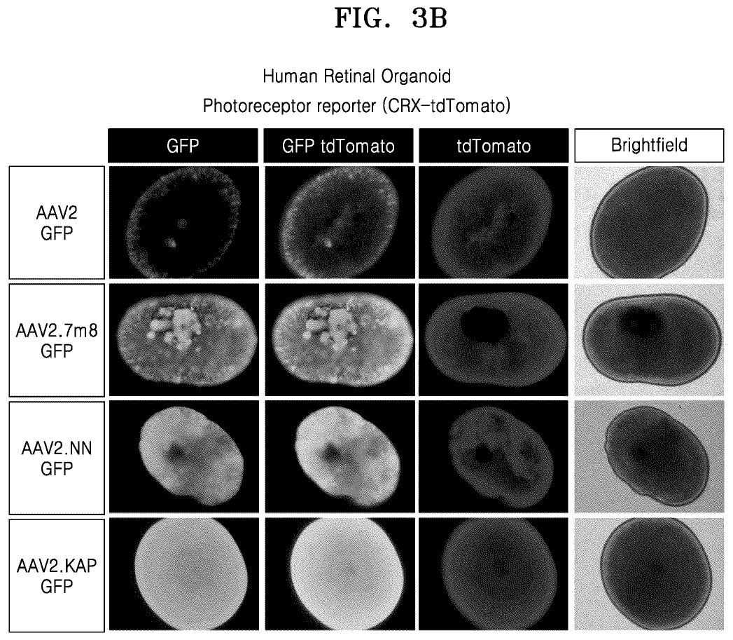

In human retinal organoids, photoreceptor cells, which are target cells of AAV2.KAP, are located at the outermost layer. When a gene expressing EGFP was delivered, it was confirmed that wild-type AAV2, as known in the art, was unable to penetrate the retinal layers and thus failed to infect the interior. In addition, in human retinal organoids treated with AAV2.KAP, regions in which tdTomato fluorescent protein expressed specifically in photoreceptor cells and EGFP fluorescent protein were merged and appeared yellow were broader than those in human retinal organoids treated with AAV2, AAV2.7m8, or AAV2.NN. Accordingly, AAV2.KAP exhibited a significantly higher EGFP gene transduction efficiency throughout the entire human retinal organoid compared to wild-type AAV2 and improved AAV2.7m8 and AAV2.NN. The results are illustrated in FIG. 3A and FIG. 3B.

It was confirmed that the AAV2.KAP variant exhibited superior gene delivery to human photoreceptor cells compared to wild-type AAV2, AAV2.7m8, and AAV2.NN.

### 2.2. Quantitative Analysis of Gene Transduction Efficiency by FACS

Gene transduction efficiency and expression efficiency of AAV2.KAP in human retinal organoids were quantitatively analyzed.

Specifically, human retinal organoids expressing tdTomato fluorescence specifically in photoreceptor cells, prepared as disclosed in 1.1, were transfected with AAV2. KAP carrying a gene encoding EGFP as a target gene, and with control group wild-type AAV2, improved AAV2.7m8, and AAV2.NN, at 5×10¹⁰ GC, and FACS analysis was performed three weeks later. Human retinal organoids were dissociated using a Papain Dissociation System (Worthington Biochemical Corporation, NJ, USA). FACS was performed using a BD FACS Aria II (BD Biosciences, CA, USA), and analysis was conducted using FlowJo software (Tree Star, OR, USA).

tdTomato fluorescence labels photoreceptor cells, and GFP fluorescence labels cells to which the target gene was delivered. Gene transduction efficiency to the entire retinal cells was analyzed as a proportion of GFP-positive cells among total cells, gene transduction efficiency to photoreceptor cells was analyzed as a proportion of GFP-positive cells among tdTomato-positive cells, and gene transduction efficiency to retinal cells other than photoreceptor cells was analyzed as a proportion of GFP-positive cells among tdTomato-negative cells.

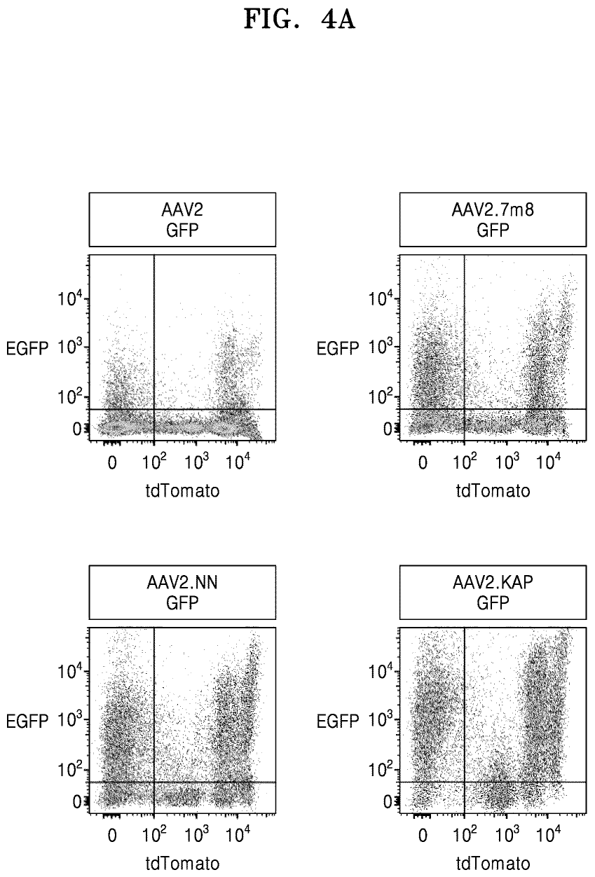

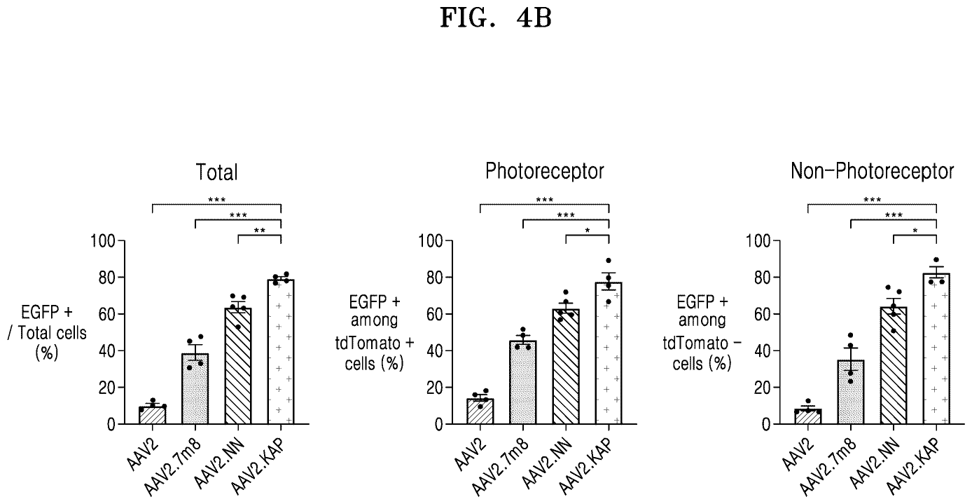

According to FACS analysis, the proportion of GFP-positive cells among total cells in human retinal organoids was 79.22±1.11% for AAV2.KAP, which was significantly higher and statistically significant compared to wild-type AAV2 (10.19±1.09%), improved AAV2.7m8 (39.08±4.26%), and AAV2.NN (63.76±3.05%). In addition, the proportion of GFP-positive cells among photoreceptor cells (tdTomato+) was 77.69±4.67% for AAV2.KAP, which was significantly higher and statistically significant compared to AAV2 (14.31±1.84%) and AAV2.7m8 (46.01±2.37%). FIG. 4A and FIG. 4B illustrate a representative image of the FACS analysis results and the quantitative analysis results, respectively.

### 2.3. Detection of Gene Expression by Immunofluorescence Staining

To confirm gene delivery to photoreceptor cells and Müller glia cells, immunofluorescence staining was performed using antibodies labeling these cells and an antibody against GFP delivered by AAV2.

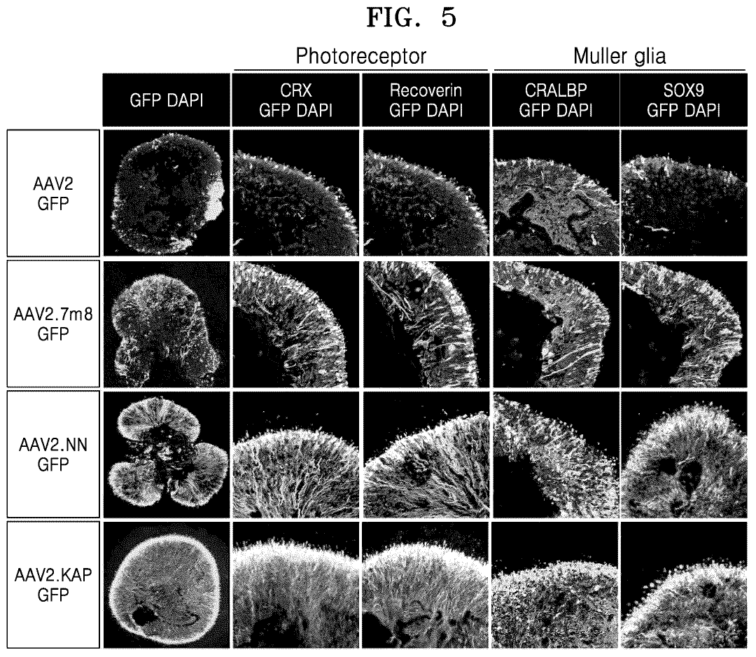

Human retinal organoids transfected with AAV2.KAP-GFP and control group wild-type AAV2-GFP, improved AAV2.NN-GFP, and AAV2.7m8-GFP were fixed for 25 minutes in a 5% sucrose solution including 4% paraformaldehyde. After fixation, the samples were sequentially treated with 10%, 20%, and 30% sucrose solutions for 1 hour each. For sectioning, human retinal organoids were embedded using an optical coherence tomography (OCT) compound and rapidly frozen using liquid nitrogen vapor. Thereafter, sections having a thickness of 7 μm were prepared using a cryostat (Leica Microsystems, Wetzlar, Germany), and differences in GFP gene expression in the human retinal organoids were examined. The results are illustrated in FIG. 5. Expression of the GFP gene delivered by AAV2 variants and wild-type AAV2 was merged with fluorescence of photoreceptor marker antibodies (CRX and Recoverin antibodies) and fluorescence of Müller glial cell marker antibodies (CRALBP and SOX9 antibodies), respectively. GFP gene expression by the AAV2.KAP variant showed broader merged regions in photoreceptor cells and Müller glia cells than those of wild-type AAV2 and improved AAV2.NN and AAV2.7m8. Accordingly, it was confirmed that the selected AAV2.KAP exhibited higher gene transduction efficiency to photoreceptor cells and Müller glia cells than wild-type AAV2 and improved AAV2.7m8 and AAV2.NN.

### Example 3. In Vivo Validation of AAV2.KAP Variants: Mice

The gene delivery capability of the AAV2.KAP variant selected in Example 1 to photoreceptor cells was confirmed in a reporter mouse (B6.Cg-Tg (Nrl-EGFP) 1Asw/J) in which rod photoreceptor cells are labeled with EGFP fluorescence.

### 3.1. Confirmation of Gene Delivery and Expression by Fluorescence Microscopy

to verify the gene transduction efficiency of AAV2.KAP in mice, AAV2.KAP carrying a gene encoding tdTomato, and as control groups AAV2, AAV2.7m8, and AAV2.NN, were each intravitreally injected at 1×10¹⁰ GC into 8-week-old mice (B6.Cg-Tg (Nrl-EGFP) 1Asw/J) expressing EGFP fluorescence in rod cells, and after 3 weeks, the mice were sacrificed and the eyeballs were extracted. The extracted eyeballs were fixed in 1% paraformaldehyde for 1 hour, and thereafter sequentially treated with 10%, 20%, and 30% sucrose solutions for 1 hour each. For sectioning, the eyes were embedded in an optical coherence tomography (OCT) compound such that the connection between the optic nerve and the corneal central junction was horizontally aligned, and were rapidly frozen using liquid nitrogen vapor. Thereafter, sections were prepared at 7 μm thickness using a cryostat (Leica Microsystems, Wetzlar, Germany). After staining with DAPI to label cell nuclei, expression of EGFP and tdTomato was imaged using a fluorescence microscope.

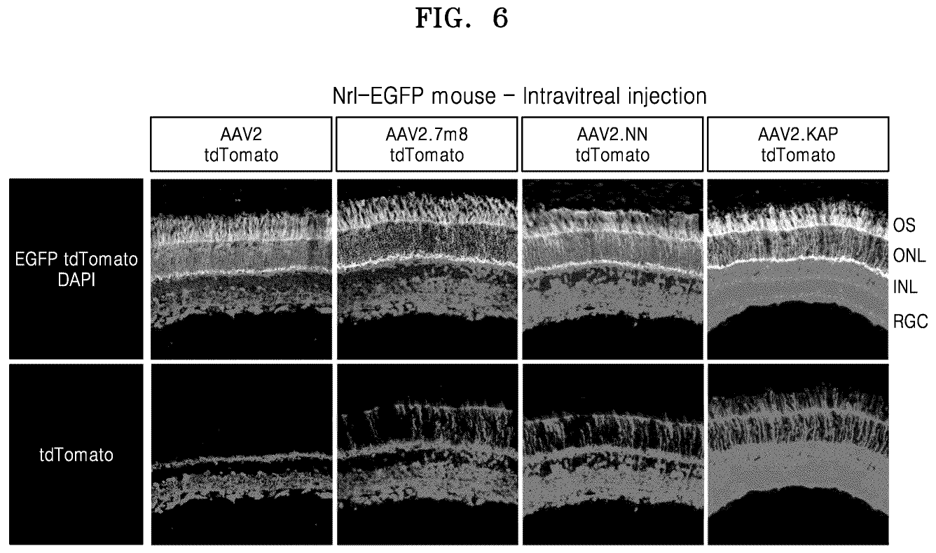

It was confirmed that wild-type AAV2 infected retinal ganglion cells (RGCs) and a portion of the inner nuclear layer (INL). In contrast, AAV2.KAP was confirmed to have significantly higher expression of the tdTomato gene delivered to not only RGCs and INL but also to the outer nuclear layer (ONL) and outer segment (OS) region, which are the nuclear layers of photoreceptor cells. The results are illustrated in FIG. 6.

### 3.2. Quantitative Analysis of Gene Transduction Efficiency by FACS

To quantify the gene transduction efficiency of AAV2.KAP in mice, AAV2.KAP carrying a gene encoding tdTomato, and as control groups AAV2, AAV2.7m8, and AAV2.NN, were each intravitreally injected at 1×10¹⁰ GC into 8-week-old mice (B6.Cg-Tg (Nrl-EGFP) 1Asw/J) expressing EGFP fluorescence in rod cells, and after 3 weeks, the mice were sacrificed and the eyeballs were extracted. The retinal tissue was dissociated into single cells using a Papain Dissociation System (Worthington Biochemical Corporation, NJ, USA), and FACS was performed using a BD FACSymphony S6 Cell Sorter (BD Biosciences, CA, USA). FACS analysis was conducted using FlowJo software (Tree Star, OR, USA).

EGFP fluorescence labels rod cells, and tdTomato fluorescence labels cells to which the gene was delivered. The gene transduction efficiency to the entire retinal cells was measured as the proportion of tdTomato-positive cells among total cells, the gene transduction efficiency to rod cells was measured as the proportion of tdTomato-positive cells among EGFP-positive cells, and the gene transduction efficiency to retinal cells excluding rod cells was measured as the proportion of tdTomato-positive cells among EGFP-negative cells.

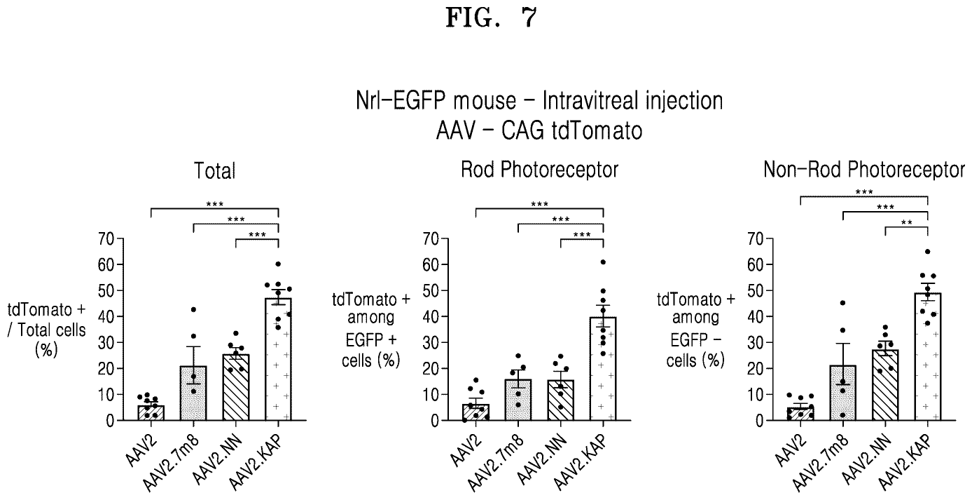

As a result of FACS analysis, the proportion of tdTomato-positive cells among total mouse retinal cells was 47.48±2.90% for AAV2.KAP, which was significantly higher and statistically significant compared to the control group wild-type AAV2 (6.18±1.09%), the improved AAV2.7m8 (21.30±7.18%), and AAV2.NN (25.84±2.24%). Additionally, the proportion of tdTomato-positive cells among rod cells (EGFP+) was 40.14±4.17% for AAV2.KAP, which was significantly higher than that of AAV2 (6.52±2.00%), AAV2.7m8 (15.95±3.42%), and AAV2.NN (15.78±3.09%), and was statistically significant. The results are illustrated in FIG. 7.

### 3.3. Detection of Gene Expression by Immunofluorescence Staining

To verify the gene transduction efficiency of AAV2.KAP, and, as control groups, wild-type AAV2 and the improved AAV2.7m8 and AAV2.NN, each carrying a gene encoding EGFP, were intravitreally injected into C57BL/6J wild-type mice.

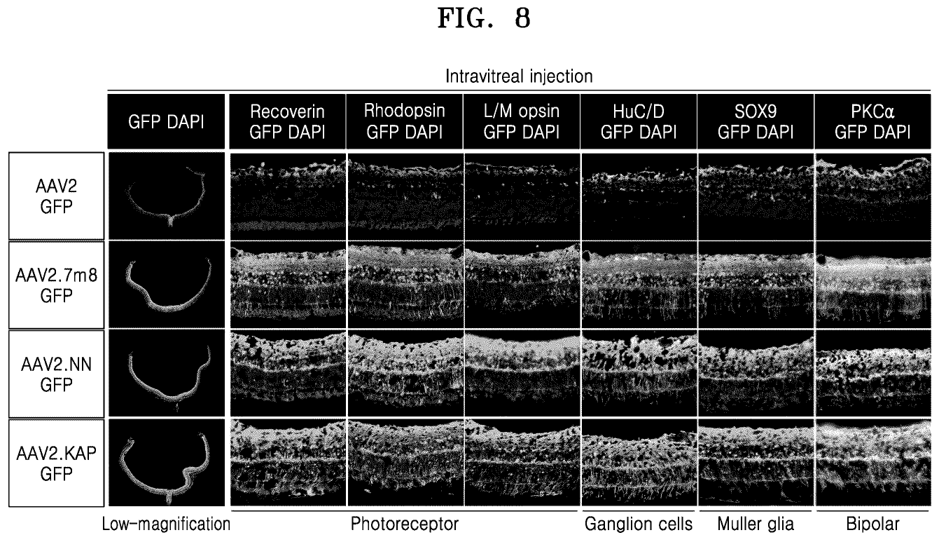

Specifically, 8-week-old C57BL/6 mice were intravitreally injected with AAV2.KAP carrying a gene encoding EGFP and, as control groups, AAV2/AAV2.7m8/AAV2.NN, each at 2×10¹⁰ GC, and, 3 weeks later, the mice were sacrificed and the eyes were extracted. The extracted eyes were fixed in 1% paraformaldehyde for 1 hour. After fixation, the samples were sequentially treated with 10%, 20%, and 30% sucrose solutions for 1 hour each. For sectioning, the eyes were embedded in an optical coherence tomography (OCT) compound such that the connection between the optic nerve and the corneal central junction was horizontally aligned, and were rapidly frozen using liquid nitrogen vapor. Thereafter, sections were prepared at 7 μm thickness using a cryostat (Leica Microsystems, Wetzlar, Germany). Immunofluorescence staining was performed on the prepared sections using antibodies labeling retinal cells (Recoverin antibody: photoreceptor cells; Rhodopsin antibody: rod photoreceptor cells; L/M opsin antibody: L/M cone photoreceptor cells; HuC/D antibody: ganglion cells; SOX9 antibody: Muller glial cells; PKCα antibody: bipolar cells) and an antibody against EGFP fluorescence delivered by AAV2. After staining with DAPI to label cell nuclei, expression of EGFP was imaged using a fluorescence microscope. The results are illustrated in FIG. 8.

For AAV2.KAP, infection spreading throughout the entire retina was confirmed in low-magnification fluorescence images. AAV2.KAP was confirmed to have higher gene transduction efficiency to photoreceptor cells, ganglion cells, Müller glial cells, and bipolar cells compared to wild-type AAV2, and higher gene transduction efficiency to photoreceptor cells compared to the improved AAV2.7m8 and AAV2.NN.

Through these results, it was confirmed that, compared with wild-type AAV2 and improved AAV2.7m8 and AAV2.NN, the KAP variant selected in Example 1 passes through the ILM from the injection site at a high rate via intravitreal injection, delivers a target gene to photoreceptor cells, and expresses the target gene at a high level.

The KAP variant according to the disclosure was confirmed to have excellent gene transduction efficiency through passing through the ILM by intravitreal injection, delivering a target gene to retinal cells including photoreceptors, inducing strong gene expression, providing increased gene expression and lateral spreading compared to wild-type AAV2, and increased photoreceptor-specific gene transduction and gene expression compared to wild-type AAV2 in human retinal organoids. Accordingly, the KAP variant according to the disclosure may be usefully employed in gene therapy for treatment of ocular diseases.

It should be understood that embodiments described herein should be considered in a descriptive sense only and not for purposes of limitation. Descriptions of features or aspects within each embodiment should typically be considered as available for other similar features or aspects in other embodiments. While one or more embodiments have been described with reference to the figures, it will be understood by those of ordinary skill in the art that various changes in form and details may be made therein without departing from the spirit and scope of the disclosure as defined by the following claims.

What is claimed is:

1. An AAV2 variant capsid protein comprising a peptide KAPPNNP inserted into a GH-loop of a wild-type AAV2 capsid protein.

2. The AAV2 variant capsid protein of claim 1, wherein the peptide is inserted at amino acid position 587 of the wild-type AAV2 capsid protein of SEQ ID NO: 1, or at a corresponding position thereof.

3. The AAV2 variant capsid protein of claim 1, wherein, as compared with a wild-type AAV2 capsid protein, the AAV2 variant capsid protein confers increased infectivity into retinal cells via intravitreal injection.

4. The AAV2 variant capsid protein of claim 3, wherein the retinal cells are selected from the group consisting of photoreceptor cells, retinal ganglion cells, bipolar cells, Müller glial cells, horizontal cells, amacrine cells, astrocytes, microglial cells, retinal vascular cells, and retinal pigment epithelial cells.

5. An isolated nucleic acid encoding the AAV2 variant capsid protein of claim 1.

6. A host cell comprising an isolated nucleic acid encoding the AAV2 variant capsid protein of claim 1.

7. A recombinant AAV vector, comprising: the AAV2 variant capsid protein of claim 1; and
a heterologous nucleic acid comprising a sequence encoding a target product.

8. The recombinant AAV vector of claim 7, wherein, as compared with an AAV vector comprising a wild-type AAV2 capsid protein, the recombinant AAV vector provides increased transduction into retinal cells via intravitreal injection.

9. The recombinant AAV vector of claim 8, wherein the retinal cells are selected from the group consisting of photoreceptor cells, retinal ganglion cells, bipolar cells, Müller glial cells, horizontal cells, amacrine cells, astrocytes, microglial cells, retinal vascular cells, and retinal pigment epithelial cells.

10. The recombinant AAV vector of claim 7, wherein the target product is a polypeptide or a nucleic acid.

11. A pharmaceutical composition for treatment of an ocular disease, comprising the recombinant AAV vector of claim 7.

12. The pharmaceutical composition of claim 11, wherein the ocular disease is selected from retina-vitreous-choroid related diseases, retinal vascular diseases, macular diseases, hereditary retinal diseases, hereditary vitreous diseases, hereditary choroidal diseases, intraocular inflammatory diseases, intraocular tumors, glaucoma, and optic neuropathies.

13. The pharmaceutical composition of claim 11, wherein the ocular disease is selected from macular degeneration, diabetic retinopathy, occlusion of retinal vessels, macular telangiectasia, retinopathy of prematurity, myopic degeneration, retinitis pigmentosa, Leber congenital amaurosis, Best's Disease, Stargardt's Disease, congenital stationary night blindness, X-linked retinoschisis, Bietti crystalline dystrophy, achromatopsia, cone dystrophy, cone-rod dystrophy, maculopathy, Usher syndrome, Bardet-Biedl syndrome, syndromic retinitis pigmentosa, pan choroidal atrophy, central areolar choroidal dystrophy, gyrate atrophy of choroid and retina, glaucoma, optic neuropathy, uveitis, uveal melanoma, intraocular lymphoma, retinoblastoma, retinoschisis, and retinal injury.

14. The pharmaceutical composition of claim 11, wherein the pharmaceutical composition is administered by intravitreal injection, subretinal injection, or suprachoroidal injection.

15. An in-vitro method of delivering a heterologous nucleic acid into retinal cells, comprising contacting the retinal cells with the recombinant AAV vector of claim 7.

16. The method of claim 15, wherein the retinal cells are selected from the group consisting of photoreceptor cells, retinal ganglion cells, bipolar cells, Müller glial cells, horizontal cells, amacrine cells, astrocytes, microglial cells, retinal vascular cells, and retinal pigment epithelial cells.


SEQUENCE LISTING (4 sequences)

SEQ ID NO: 1 - protein, 735 aa, Adeno-associated dependoparvovirus A [sequence omitted]

SEQ ID NO: 2 - protein, 742 aa, synthetic construct
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPGKKRPVEHSPVEPDSSSGTGKAGQQPARKRLNFGQTGDADSVPDPQPLGQPPAAPSGLGTNTMATGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTPSGTTTQSRLQFSQAGASDIRDQSRNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQRGNKAPPNNPRQAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

SEQ ID NO: 3 - protein, 745 aa, synthetic construct
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPGKKRPVEHSPVEPDSSSGTGKAGQQPARKRLNFGQTGDADSVPDPQPLGQPPAAPSGLGTNTMATGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTPSGTTTQSRLQFSQAGASDIRDQSRNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQRGNLALGETTRPARQAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

SEQ ID NO: 4 - protein, 747 aa, synthetic construct
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPGKKRPVEHSPVEPDSSSGTGKAGQQPARKRLNFGQTGDADSVPDPQPLGQPPAAPSGLGTNTMATGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTPSGTTTQSRLQFSQAGASDIRDQSRNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQRGNAAANNPTPSRAARQAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL